(19) United States
(12) Patent Application Publication
(10) Pub. No.: US 2015/0094447 A1
(43) Pub. Date: Apr. 2, 2015

(54) Novel Nitric Oxide Synthase Agonist Polymers

(71) Applicant: Wayne H. KAESEMEYER, Chapel Hill, NC (US)

(72) Inventor: Wayne H. KAESEMEYER, Chapel Hill, NC (US)

(73) Assignee: PALMETTO PHARMACEUTICALS LLC, Charleston, SC (US)

(21) Appl. No.: 14/541,961

(22) Filed: Nov. 14, 2014

Related U.S. Application Data
(63) Continuation-in-part of application No. 13/426,948, filed on Mar. 22, 2012, now Pat. No. 8,936,635.
(60) Provisional application No. 61/457,419, filed on Mar. 23, 2011.

(51) Int. Cl. A61K 31/765; A61K 31/785; C08G 63/08; A61K 31/795; C08G 63/685; C08G 63/688

(52) U.S. Cl. CPC A61K 31/765; C08G 63/685; C08G 63/6882; C08G 63/08; A61K 31/795; A61K 31/785
USPC 528/290; 528/298; 528/291; 528/331; 528/288; 528/321; 528/354

(57) ABSTRACT

The invention relates to polymers comprising NOS agonists and one or more monomers selected from the group consisting of lactide, glycolide and epsiloncaprolactone, wherein the NOS agonist is incorporated as repeating monomer units into the body or backbone of the polymer are disclosed. The NOS agonist may comprise one or more carboxylic acid groups and one or more alkylhydroxy groups. In another embodiment, the NOS agonist may comprise a carboxylic acid group and an alkylhydroxy group that can be joined to form a lactone containing cyclic ring. In another embodiment, the NOS agonist comprises HMG CoA reductase inhibitor or statin.

The present application is a continuation in part of application Ser. No. 13/426,948, filed Mar. 22, 2012, which claims benefit under 35 U.S.C. 119(e) of Provisional Application No. 61/457,419, filed Mar. 23, 2011, the disclosures of which are expressly incorporated by reference herein in their entirety.

### RELATED APPLICATION

Ghatnekar et al., application Ser. No. 13/612,054, filed Sep. 12, 2012, now abandoned, the disclosure of which is expressly incorporated by reference herein in its entirety, teach a bioresorbable polymer comprising statin and one or more monomers selected from the group consisting of lactide, glycolide and lactone, wherein the statin is incorporated as repeating monomer units into the body of the polymer.

### FIELD OF THE INVENTION

The present invention relates to polymers comprising nitric oxide agonists and one or more monomers selected from the group consisting of lactide, glycolide and lactone, wherein the nitric oxide agonist is incorporated as repeating monomer units into the body or backbone of the polymer are disclosed. The nitric oxide agonist may have a lactone ring. Other nitric oxide agonist may have a carboxylic acid group and alkylhydroxy groups. In another embodiment, the nitric oxide agonist may have a carboxylic acid and alkylhydroxy group that can be joined to form a lactone containing cyclic ring by condensation. The nitric agonist may also be a statin or HMG CoA reductase inhibitor. These polymers have intrinsic pharmacologic bioactivity. According to embodiments of the invention, they can accomplish one or more of the following: increase nitric oxide, prevent endothelial dysfunction induced by antirestenosis agents used in drug eluting stents, promote re-endothelization, reduce the risk of stent thrombosis and the requirement for antiplatelet drug therapy and its risk of bleeding, and related morbidity and mortality, following procedure related vascular lumen injury. When used in surgical devices such as sutures, meshes, implants, bone grafts, etc., the nitric oxide agonists released from the polymers upon their bioresorption reduce inflammation, promote wound healing and reduce scarring.

### BACKGROUND OF THE INVENTION

Recently a stent scaffold has been constructed from a polylactide polymer typically used to make bioresorbable sutures and other related bioresorbable surgical devices. See Capodanno et al., “Novel Drug-eluting Stents in the Treatment of de novo Coronary Lesions”, Vascular Health and Risk Management, vol. 7, pp 103-118 (2011). This scaffold has been used to fabricate a biodegradeable polymer stent upon whose surface everolimus, an antirestenosis agent, is coated.

Polylactide is a terpolymer of (a) L(-)lactide, (b) glycolide, and (c) epsilon-caprolactone. The L(-) lactide component varies from 45-85% by weight. Glycolide varies from 5-50% by weight. And the epsilon-caprolactone varies from about 15 to 25% by weight. The initial use of polylactide in bioresorbable stent construction was for urethral stents. See Goldberg et al., U.S. Pat. No. 5,085,629.

The process of synthesizing polylactide involves the presence of a lactone ring in epsilon-caprolactone. HMG CoA reductase inhibitors, or statins, likewise have structures that can exist in a form with a closed lactone ring. It has recently been shown that simvastatin can be embedded into microspheres of DL-lactide-glycolide to form simvastatin containing microspheres which can be placed in a PolyRing for sustained perivascular drug delivery to vascular grafts and dialysis access devices to prevent intimal hyperplasia. See Krishnan, “Simvastatin Incorporated Perivascular Polymeric Controlled Drug Delivery System for the Inhibition of Vascular Wall Intimal Hyperplasia,” A Thesis Presented to The Graduate Faculty at The University of Akron. August, 2007. The polymer of the Krishnan microspheres differs from the polymer of the present invention in that the simvastatin of the Krishnan microspheres is not chemically incorporated into the body or backbone of the polymer.

A bioabsorbable polymer scaffold for a stent is taught by Johnson et al., US published Application No. 2008/0249608. The scaffold comprises a plurality of hoop components configured as the primary radial load bearing elements of the intraluminal scaffold; and one or more connector elements interconnecting the plurality of hoop components, wherein at least one of the plurality of hoop components and the one or more connector elements comprises a composite structure formed from a bioabsorbable metallic material and a bioabsorbable polymeric material. Although Johnson describes a plurality of therapeutic and pharmaceutic agents including a statin can be coated on the scaffold, the Johnson scaffold does not include a statin incorporated as repeating monomer units into the backbone of the polylactide polymeric scaffold, as taught according to embodiments of the present invention. And upon bioresorpton of the Johnson et al scaffold, the added drugs can exert their separate pharmacologic bioactivities but, overall, the scaffold illustrates extrinsic pharmacologic bioactivity instead of the intrinsic pharmacologic bioactivity seen with the scaffold of the present invention. And this is because the Johnson scaffold, minus the effects of the drugs added to the scaffold, has no “intrinsic” pharmacologic activity and does not function as a prodrug during the process of bioresorption, as does the scaffold described herein. Lastly, unlike the Johnson scaffold, the pharmacologically active agents in the scaffold of this invention are chemically bonded as monomers in the overall polymeric structure of the bioresorbable scaffold.

A bioresorbable polystatin for use in constructing scaffolds for vascular stents is attractive because with metabolism of the polystatin to its monomeric form which occurs in the bioresorption process, the action of the released statin to increase the activity of nitric oxide synthase is felt to reduce the risk of stent thrombosis and the need for post stent antiplatelet therapy with its risk of bleeding. See Kaesemeyer, “Statin DES for Early Stent Thrombosis,” Atherosclerosis. 207: 343 (2009); Kaesemeyer, U.S. Pat. No. 6,425,881; Jaschke et al., “Local Statin Therapy Differentially Interferes with Smooth Muscle and Endothelial Proliferation and Reduces Neointima on a Drug-Eluting Stent Platform,” Cardiovas Res, 68: 483-92(2005); and Miyauchi et al., “Effectiveness of Statin-Eluting Stent on Early Inflammatory Response and Neointimal Thickness in a Porcine Coronary Model,” Circ J. 72: 832-8 (2008). The same principal applies to any nitric oxide agonist possessing a lactone ring in its structure that will permit polylactide polymer formation.

Lim et al., US published Application No. 2009/0306120, discloses an amorphous terpolymer comprising lactide, glycolide and caprolactone, which can be a coating on an implantable device for controlling the release of drugs or can be a bioabsorabable implantable device such as a bioabsorbable stent. Lim et al. disclose lists of biologically active agents including lovastatin (a drug that inhibits HMG-CoA reductase). Lim et al. does not teach a scaffold comprising a L(-) lactide-glycolide-statin terpolymer according to embodiments of the present invention. And upon bioresorpton of the Lim et al scaffold, the added drugs can exert their separate pharmacologic bioactivities but, overall, the scaffold illustrates extrinsic pharmacologic bioactivity instead of the intrinsic pharmacologic bioactivity seen with the scaffold of the present invention. The Lim scaffold, minus the effects of the drugs added to the scaffold, has no “intrinsic” pharmacologic activity and does not function as a prodrug during the process of bioresorption, as does the scaffold described herein. Lastly, unlike the Lim scaffold, the pharmacologically active agents in the scaffold of this invention are chemically bonded as monomers in the overall polymeric structure or backbone of the bioresorbable scaffold.

### SUMMARY OF THE INVENTION

The present invention relates to a polymer comprising a therapeutic agent capable of being polymerized and one or more monomers selected from the group consisting of lactide, glycolide and lactone, wherein the therapeutic agent is incorporated as repeating monomer units into the body or backbone of the polymer.

The therapeutic agent may be a NOS (nitric oxide synthase) agonist. In one embodiment of the present invention, the NOS agonist may have a lactone ring. In another embodiment, the NOS agonist may have one or more carboxylic acid groups and one or more alkylhydroxy groups. In another embodiment, the NOS agonist may have a carboxylic acid group and alkylhydroxy group that can be joined to form a lactone containing cyclic ring by condensation. The NOS agonist may also be a statin or HMG CoA reductase inhibitor. The NOS agonist having a lactone ring may be incorporated into the polymer as a result of undergoing a ring opening polymerization in a similar way as does epsilon-caprolactone.

In another embodiment of the present invention, the polymer may comprise NOS agonist and one or more monomers selected from the group consisting of lactide, glycolide, and lactone, wherein the NOS agonist is incorporated as repeating monomer units into the body of the polymer. The NOS agonist may have one or more carboxylic acid groups and one or more hydroxy groups. In another embodiment, the NOS agonist may have a carboxylic acid group and a hydroxy group that can be joined to form a lactone containing cyclic ring by condensation. The NOS agonist may be HMG CoA reductase inhibitor or statin. The statin may be selected from the group consisting of velostatin, dihydrocompactin, carvastatin, bevastatin, cefvastatin, glenvastatin, simvastatin, lovastatin, atorvastatin, pravastatin, cerivastatin, rosuvastatin, pitavastatin, fluvastatin, mevastatin, dalvastatin, compactin and mixtures thereof.

The polymer may be selected from the group consisting of polylactide NOS agonist, polyglycolide NOS agonist, polylactone NOS agonist, polylactideglycolide NOS agonist, polylactidelactone NOS agonist, polyglycolidelactone NOS agonist, polylactideglycolide lactone NOS agonist and mixtures thereof.

The polymer may comprise NOS agonist and one or more monomers selected from the group consisting of lactide, glycolide, and epsilon caprolactone, wherein the NOS agonist is incorporated as repeating monomer units into the body of the polymer. The NOS agonist may have one or more carboxylic acid groups and one or more hydroxy groups. In another embodiment, the NOS agonist may have a carboxylic acid group and a hydroxy group that can be joined to form a lactone containing cyclic ring by condensation. The NOS agonist may be HMG CoA reductase inhibitor or statin. The statin may be selected from the group consisting of velostatin, dihydrocompactin, carvastatin, bevastatin, cefvastatin, glenvastatin, simvastatin, lovastatin, atorvastatin, pravastatin, cerivastatin, rosuvastatin, pitavastatin, fluvastatin, mevastatin, dalvastatin, compactin and mixtures thereof.

The polymer may be selected from the group consisting of polylactide NOS agonist, polyglycolide NOS agonist, polyepsilon caprolactone NOS agonist, polylactideglycolide NOS agonist, polylactideepsilon caprolactone NOS agonist, polyglycolideepsilon caprolactone NOS agonist, polylactideglycolide epsiloncaprolactone NOS agonist and mixtures thereof. The statin may be selected from the group consisting of velostatin, dihydrocompactin, carvastatin, bevastatin, cefvastatin, glenvastatin, simvastatin, lovastatin, atorvastatin, pravastatin, cerivastatin, rosuvastatin, pitavastatin, fluvastatin, mevastatin, dalvastatin, compactin and mixtures thereof.

In another embodiment of the present invention, the polymer may be selected from the group consisting of polylactide statin, polyglycolide statin, polyepsilon caprolactone statin, polylactide-glycolide-statin, polylactide-epsilon caprolactone-statin, polyglycolide-epsilon caprolactone-statin, polylactide-glycolide-epsilon-caprolactone statin and mixtures thereof. The statin may be in the range from about 1 to about 99 weight percent.

Embodiments of the present invention are directed to a resorbable biomaterial including polymeric statins, and methods for using these resorbable biomaterials, including to promote wound healing, reduce scaring, reduce bleeding, and mitigate post-surgical adhesions. In certain embodiments, bioresorbable therapeutically active polymers comprising statin monomers covalently incorporated into the backbone of polymer scaffolds are disclosed. The polystatin polymers of embodiments may be synthesized from L (-) lactide, glycolide, and a lactone containing statin with an intact lactone ring. In yet other embodiments, everolimus and zotarolimus and other thrombosis reducing drugs are incorporated into the disclosed devices thereby reducing bleeding following device placement and reducing duration and intensity of dual anti platelet therapy (DAPT) currently required for PCI. The dissolution of the biomaterial when implanted in the injured tissues or wounds may enable the controlled release of the biomaterial to exhibit its pharmacological properties as a drug or prodrug, preferably at least one statin, most preferably a statin possessing plieotropic effects. In addition, the biomaterial may be coated with other pharmacologically active agents, including but not limited to, nitric oxide agonists, antimicrobials, growth factors, anti-inflammatory agents. In some embodiments, the polystatin polymer is extruded to form flexible thin filaments that can be configured to form implants, including but not limited to stents, sutures, membranes, meshes, grafts, synthetic tendons, synthetic ligaments, or the like. In certain embodiments, these polymers are extruded into a scaffold for use as a vascular stent. Additional objects, features, embodiments, and advantages of the invention will become more apparent upon review of the detailed description set forth below.

### DETAILED DESCRIPTION OF THE INVENTION

Below are definitions for terms related to the present invention. In describing and claiming the present invention, the following terminology will be used in accordance with the definitions set out below. Notwithstanding, other terminology and definitions may also be found throughout this disclosure as well.

Bioresorbable—the materials that can be broken down by the body and that do not require removal, such as sutures or the chlorhexidine chip.

Biodegradable—capable of being broken down especially into innocuous products by the action of living things (such as microorganisms). The antirestenosis drug containing polymers used to coat the outside surface of scaffolds of drug-eluting stents are biodegradable. Once the drug is eluted the polymer carrying the drug is biodegraded

Regarding the term “bioresorbable” as it applies to a bioresorbable article, including a scaffold, the article does not require removal because it is reabsorbed into the surrounding vasculature when it is broken down. The term biodegradable, on the other hand, would only require the article to be broken down by biological processes without specifying the final fate of the products of the decomposition process.

### Utility

Embodiments of the invention include resorbable biomaterials, and methods for using the restorable biomaterials, and in certain embodiments, the resorbable biomaterial may be incorporated into the fabrication of implants including, but not limited to stents, sutures, membranes, meshes, grafts, and the like. The resorbable biomaterials in such embodiments generally include polystatin polymers and are pharmacologically active for stimulating tissue repair, wound closure, and generally promote wound healing of tissues, organs, surgical wounds, surgical incisions, and the like when administered at or adjacent to the wound. In some embodiments, wounds treated with the resorbable biomaterials of the invention exhibit reduced scar formation and tissue adhesion. In certain other embodiments, the biomaterials prevent excessive bleeding and thrombosis.

Nitric Oxide Agonists.

Unlike nitric oxide donors which release nitric oxide from the donor molecule itself, NOS agonists are agents that increase or upregulate nitric oxide production from the enzyme nitric oxide synthase (NOS). NOS upregulation involves increases in NOS activity or gene expression produced by agents referred to as NOS agonists. Other types of nitric oxide agonists include, but are not limited to, NOS substrate (L-arginine), NOS cofactor (tetrahydrobiopterin—BH4) and L-arginine equivalents. See Kaesemeyer, U.S. Pat. No. 5,767,160. A gene for NOS and related genetic materials can also function as a nitric oxide agonist when it is transfected into the lumenal wall of blood vessels.

Examples of L-arginine equivalents include, but are not limited to, citrulline, arginase inhibitors and the antioxidants n-acetyl-L-cysteine, ascorbic acid, tempol, hydralazine, and pentaerythritol tetranitrate (PETN). Citrulline functions as a prodrug which is converted to L-arginine. Arginase inhibitors block the shunting of L-arginine into the orthnithine cycle and thereby increase the availability of L-arginine to NOS. The antioxidants prevent oxidative injury to the CAT 1 or y+ transporter which provides for the uptake of L-arginine into the endothelial cell and its delivery to NOS which utilizes L-arginine to produce (endothelium derived) nitric oxide.

NOS agonists can be either physiologic or pharmacologic. Examples of physiologic agonists of NOS include, but are not limited to, acetylcholine, bradykinin, histamine, serotonin and substance P. Pharmacologic NOS agonists include, but are not limited to, inhibitors of converting enzyme or kinase II, angiotensin receptor blockers, endothelin antagonists, certain beta blockers such as nevibilol and HMG CoA reductase inhibitors or statins. NOS agonists, especially statins, have the ability to acutely activate NOS. See Kaesemeyer et al., “Pravastatin Sodium Activates Endothelial Nitric Oxide Synthase Independent of its Cholesterol Lowering Actions,” 33:234-41 (1999); and Datar et al., “Acute Activation of eNOS by Statins Involves Scavenger Receptor—B1, G-Protein Subunit Gi Phospholipase C and Calcium Influx,” Br J Pharmacol.160: 1765-1772 (2010). Because of their ability to acutely activate NOS and promote re-endothelialization, in addition to having structures possessing lactone rings, statins are felt to be ideally suited for preventing stent thrombosis. See Kaesemeyer, “Statin DES for Early Stent Thrombosis,” Atherosclerosis. 207: 343 (2009). However, it is conceivable that other nitric oxide agonists mentioned above such as L-arginine, BH4, L-arginine equivalents and transfected NOS gene and related genetic materials, could be used alone or modified so as to function as prodrugs containing lactone rings suitable for forming polylactide polymers with nitric oxide enhancing capabilities similar to statins.

NOS agonists may contain a lactone ring or may contain carboxylic acid groups and hydroxy groups. If the NOS agonist contains a lactone ring, the NOS agonist may be incorporated into the polymer as a result of undergoing a ring opening polymerizarion in a similar way as does epsilon-caprolactone.

Statins

Statins are a group of 3-hydroxy-3-methylglutaryl Coenzyme A reductase inhibitors in cholesterol biosynthesis pathway that have been widely used as a cholesterol lowering drug. However, statins have also has been shown to reduce hypertrophic scar formation by inhibiting Connective Tissue Growth Factor (CTGF) when administered at low doses. Similarly, lovastatin and atorvastatin have been administered with the peritoneum to up regulate local fibrinolysis decreasing post operative adhesions. Statins acutely and directly activate endothelial nitric oxide synthase (eNOS) independently of inhibiting HMG Co A Reductase. More recently this pathway has been shown to involve the SR-BI receptor on the endothelium.

There are a number of statins that are available and approved for use. These include, but are not limited to, mevastatin, lovastatin, pravastatin, simvastatin, velostatin, dihydrocompactin, fluvastatin, atorvastatin, dalvastatin, carvastatin, cerivastatin, bevastatin, cefvastatin, rosuvastatin, pitavastatin, compactin, and glenvastatin. Preferred statins are simvastatin, lovastatin, atorvastatin, pravastatin, cerivastatin, rosuvastatin, pitavastatin, fluvastatin, mevastatin, dalvastatin, compactin.

The statin compounds are administered in regimens and at dosages known in the art. Especially preferred statins include fluvastatin, atorvastatin, simvastatin, pravastatin, pitavastatin and cerivastatin.

For example, fluvastatin sodium, marketed by Novartis Pharmaceuticals as Lescol is recommended for a 20-80 mg daily oral dose range, preferably between 20 and 40 mg/day for the majority of patients. 20 to 40 mg daily doses are preferably taken once daily at bedtime. 80 mg daily doses is prescribed as 40 mg doses b.i.d. and recommended only for those individuals in which the 40 mg daily dose is inadequate to lower LDL levels satisfactorily.

Atorvastatin, marketed by Pfizer as Lipitor, has a recommended starting daily dose of 10 mg once daily, with an overall daily dose range of from 10 to 80 mg.

Simvastatin, marketed by Merck & Co., Inc., may be administered with a starting dose of 20 mg once a day in the evening, or a 10 mg dose per day for those requiring only a moderate reduction in LDL levels. The recommended overall daily dosage range taken as a single evening dose is from 5 to 80 mg.

Pravastatin sodium, marketed as Pravachol.™. by Bristol-Meyers Squibb, has a recommended starting dose of 10 or 20 mg per day, taken daily as a single dose at bedtime, with a final overall daily range of from 10 to 40 mg. Lovastatin, marketed by Merck & Co. as Mevacor.™., has a recommended daily starting dosage of 20 mg per day taken with the evening meal. The recommended final daily dosage range is from 10 to 80 mg per day in single or divided doses.

Pitavastatin, the most recently approved drug in this class, is administered in a dose range of between 1-4 mg per day. In general, all of these statins are formulated in a simple delivery system which involves immediate-release kinetic profiles of the statin monomer.

Cerivastatin, marketed as Baycol by Bayer A.G. is administered in a dose ranging from 0.3 mg to 0.8 mg daily.

All of the above statins can be made to exist in both closed or open lactone ring forms. In order to form a polylactide polystatin polymer one would want to use a statin with an intact lactone ring such as lovastatin or simvastatin as described below.

Certain statins such as, for example, simvastatin and lovastatin include a lactone containing cyclic ring, and other statins include carboxylic acids and alcohol groups that can be joined to form a lactone containing cyclic ring by condensation. By virtue of the lactone, such statins may be incorporated into the main chain of the biocompatible polymers during the ring opening polymerization process. Thus, as hydrolysis breaks down the biocompatible polymer the statin incorporated into the main chain is released and is free to stimulate healing.

Simvastatin has a lactone ring and may be incorporated into a bioresorbable polymer during ring opening polymerization, which is well known in the art as shown by Albertsson et al., Biiomacromolecules 2003, 4, 1466-1486.

Polylactide Terpolymers

Polylactide is a terpolymer of (a) L(-)lactide, (b) glycolide, and (c) epsilon-caprolactone. Typically, the L(-) lactide component varies from 45-85% by weight. Glycolide varies from 5-50% by weight. And the e-caprolactone varies from about 15 to 25% by weight. The process of synthesizing polycaprolactone requires the presence of a lactone ring in epsilon-caprolactone. HMG CoA reductase inhibitors, or statins, likewise have structures that can exist in a form with a closed lactone ring.

The polymers of the present invention comprise L(-) lactide, glycolide, and lactone.

The method of making L(-) lactide-glycolide-epsilon-caprolactone terpolymers is described by Goldberg, U.S. Pat. No. 5,085,629, whose disclosure is expressly incorporated by reference herein in its entirety.

Generally, the amount of L(-)lactide is in the range of from about 45 to about 85 weight %, preferably about 55 to about 75 weight % and most preferably about 60 to about 70 weight % of the polymer composition.

Generally, the amount of glycolide is in the range of from about 5 to about 50 weight %, and preferably about 10 to 30 weight % of the polymer composition.

Generally the amount of lactone is in the range of from about 15 and about 25% by weight of the polymer composition.

A L(-) lactide-glycolide-simvastatin terpolymer (polysimvastatin) is formed in accordance with the method described by Goldberg, U.S. Pat. No. 5,085,629. In the preparation of polysimvastatin, simvastatin substitutes as the lactone ring containing substrate for epsilon caprolactone to form a terpolymer. Thus, in the present invention the scaffold itself is a drug, or more precisely, a prodrug from which a drug is released when the scaffold is bioresorbed.

Also, in another embodiment, a NOS agonist may be incorporated as repeating monomer units into the statin polymers described herein, i.e., polylactide statin, polyglycolide statin, polyepsilon caprolactone statin, polylactide-glycolide-statin, polylactide-epsilon caprolactone-statin, polyglycolide-epsilon caprolactone-statin, polylactide-glycolide-epsilon-caprolactone statin and mixtures thereof. The NOS agonist may also be incorporated as repeating monomer units into a L(-) lactide-glycolide-simvastatin terpolymer matrix. Thus, the NOS agonist can be co-released when the scaffold is degraded and bioresorbed. The NOS agonist may be any of those as listed above and preferably can be an L-arginine equivalent or a gene for NOS. BH4 and NOS substrate (L-arginine) can also be used. The nitric oxide agonist serves to potentiate the effect of the statin in the scaffold during the process of bioresorption.

The resulting terpolymer is then extruded into the formation of a scaffold for use in vascular stent construction. The stent may be “bare”, i.e. scaffold used alone, or may have drugs applied directly or have a polymer applied to this scaffold which contains drugs for elution which prevent smooth muscle proliferation, neointimal hyperplasia and restenosis. In this later case one would have a stent capable of delivering two drugs—one targeting the endothelium and the risk of stent thrombosis and the other targeting the risk of restenosis to the site of procedure related vascular lumen injury. This may markedly reduce the requirement for antiplatelet therapy post procedure and the risk of bleeding and death from bleeding. The stent so formed is suitable for use in carotid, intracranial, aorta (coarction repair), subclavian, peripheral, renal and coronary arteries, as well as in veins such as veins of the lower extremities. Preferably, the stent is used in coronary arteries for the treatment of occlusions from atherothrombosclerotic diseases.

The amount of each monomeric unit in such embodiments can vary and may depend on the desired properties for the statin containing polymer. In some embodiments, statin containing polymers may include one or more statins that are polymerized with a single monomer, such as, lactide, glycolide, or e-caprolactone to create a statin/lactide, glycolide, or e-caprolactone copolymer. In such embodiments, the statin may be about 0.5 wt % to about 50 wt. % of the total copolymer, and the lactide, glycolide, or e-caprolactone may make up the remainder of the copolymer (i .e., about 50 wt. % to about 99.5 wt. % of the total polymer.

For example, in a terpolymer comprising simvastatin, lactide and glycolide, simvastatin may be about 0.5 wt % to about 50 wt. % of the total copolymer, and the concentration of the remaining monomers may be determined based on the concentration of monomers in known copolymers.

In some embodiments, a statin containing polymer prepared from a terpolymer of, for example, lactide, glycolide, and a lactone containing statin may include lactide at a concentration of about 45 wt. % to about 85 wt. % of the total polymer, glycolide about 5 wt. % to about 50 wt. % of the total polymer, and lactone containing statin may, generally, be about 5 wt. % to about 30 wt. % of the total polymer.

In other exemplary embodiments, the lactide may be about 55 wt. % to about 75 wt. % or about 60 wt. % to about 70 wt. % of the total polymer, the glycolide may be about 10 wt. % to about 30 wt. % of the total polymer, and the lactone containing statin may be about 15 wt. % to about 25 wt. % of the total polymer.

In all such embodiments, the skilled artisan may introduce more or less statin to provide resorbable biomaterials having different dosage.

In various embodiments, the ratio of statin, lactide and glycolide in the terpolymer may be about 70-15-15, about 70-10-20, about 70-20-10, about 80-10-10, about 80-15-5, about 80-5-15, about 60-20-20, about 60-10-30, about 60-30 10, about 60-15-25, about 60-25-15, about 90-5-5, or any value in ranges thereof.

In some embodiments, statin containing polymers produced to incorporate the statin into the main chain of a biocompatible polymer can be combined with a second biocompatible polymer that does not include a statin to create a polymer blend. For example, in various embodiments, a statin containing polymer prepared as described herein may be combined with commercially available biocompatible polymers such as those described herein that have not been modified in any way. Without wishing to be bound by theory, including unmodified biocompatible polymers with the statin containing biocompatible polymer may produce a polymer blend with physical properties that are at least comparable to the unmodified biocompatible while still providing local deliver of the statin to the wound. Such blends may be prepared at any ratio and may include two or more unmodified biocompatible polymers and/or two or more statin containing biocompatible polymers. For example, in some embodiments the ratio of unmodified biocompatible polymers to statin containing polymers may be 1:1, 2:1, 3:1, 5:1, 10:1, 20:1, 50:1, 1:2, 1:3, 1:5, 1:10, 1:20, 1:50 or any ratio between any two of these values.

Additional Polymers Useful in the Present Invention

In addition to the above described terpolymers, the present invention also provides for the following copolymers and terpolymers incorporating statins:

Polylactide Statin Copolymer. Generally, the amount of the lactide component may range from about 1 to about 99 weight percent with the amount of the statin component of the copolymer ranging from about 99 to about 1 weight percent. The polylactide statin copolymer does not contain any glycolide and epsilon-caprolactone. Thus, the polylactide statin copolymer ranges from about 1:99 weight percent of lactide to statin (1 weight percent of lactide to 99 weight percent statin), about 10:90 weight percent of lactide to statin, about 20:80 weight percent of lactide to statin, about 30:70 weight percent of lactide to statin, about 40:60 weight percent of lactide to statin, about 50:50 weight percent of lactide to statin, about 60:40 weight percent of lactide to statin, about 70:30 weight percent of lactide to statin, about 75:25 weight percent of lactide to statin, about 80:20 weight percent of lactide to statin, about 90:10 weight percent of lactide to statin, and about 99:1 weight percent of lactide to statin.

Polyglycolide Statin Copolymer. Generally, the amount of glycolide component ranges from about 1 to about 99 weight percent with the amount of the statin component of the copolymer ranging from about 99 to about 1 weight percent. The polyglycolide statin copolymer does not contain any lactide and epsilon-caprolactone. Thus, the polyglycolide statin polymer ranges from about 1:99 weight percent of glycolide to statin, about 10:90 weight percent of glycolide to statin, about 20:80 weight percent of glycolide to statin, about 30:70 weight percent of glycolide to statin, about 40:60 weight percent of glycolide to statin, about 50:50 weight percent of glycolide to statin, about 60:40 weight percent of glycolide to statin, about 70:30 weight percent of glycolide to statin, about 75:25 weight percent of glycolide to statin, about 80:20 weight percent of glycolide to statin, about 90:10 weight percent of glycolide to statin, and about 99:1 weight percent of glycolide to statin.

Polylactone Statin Copolymer. Generally, the lactone component ranges from about 1 to about 99 weight percent with the amount of the statin component of the copolymer ranging from about 99 to about 1 weight percent. The polylactone statin copolymer does not contain any lactide and glycolide. Thus, the polylactone statin copolymer ranges from about 1:99 weight percent of lactone to statin, about 10:90 weight percent of lactone to statin, about 20:80 weight percent of lactone to statin, about 30:70 weight percent of lactone to statin, about 40:60 weight percent of lactone to statin, about 50:50 weight percent of lactone to statin, about 60:40 weight percent of lactone to statin, about 70:30 weight percent of lactone to statin, about 75:25 weight percent of epsilon-caprolactone to statin, about 80:20 weight percent of epsilon-caprolactone to statin, about 90:10 weight percent of epsilon-caprolactone to statin, and about 99:1 weight percent of epsilon-caprolactone to statin.

Polyepsilon-Caprolactone Statin Copolymer. Generally, the epsilon-caprolactone component ranges from about 1 to about 99 weight percent with the amount of the statin component of the copolymer ranging from about 99 to about 1 weight percent. The polyepsilon-caprolactone statin copolymer does not contain any lactide and glycolide. Thus, the polyepsilon-caprolactone statin copolymer ranges from about 1:99 weight percent of epsilon-caprolactone to statin, about 10:90 weight percent of epsilon-caprolactone to statin, about 20:80 weight percent of epsilon-caprolactone to statin, about 30:70 weight percent of epsilon-caprolactone to statin, about 40:60 weight percent of epsilon-caprolactone to statin, about 50:50 weight percent of epsilon-caprolactone to statin, about 60:40 weight percent of epsilon-caprolactone to statin, about 70:30 weight percent of epsilon-caprolactone to statin, about 75:25 weight percent of epsilon-caprolactone to statin, about 80:20 weight percent of epsilon-caprolactone to statin, about 90:10 weight percent of epsilon-caprolactone to statin, and about 99:1 weight percent of epsilon-caprolactone to statin.

Polylactide Glycolide Statin Terpolymer. Generally, the lactide and glycolide component ranges from about 1 to about 99 weight percent with the amount of the statin component of the terpolymer ranging from about 99 to about 1 weight percent. The polylactide glycolide statin terpolymer does not contain any epsilon-caprolactone. Thus, the polylactide glycolide statin terpolymer ranges from about 1:99 weight percent of lactide and glycolide component to statin, about 10:90 weight percent of lactide and glycolide component to statin, about 20:80 weight percent of lactide and glycolide component to statin, about 30:70 weight percent of lactide and glycolide component to statin, about 40:60 weight percent of lactide and glycolide component to statin, about 50:50 weight percent of lactide and glycolide component to statin, about 60:40 weight percent of lactide and glycolide component to statin, about 70:30 weight percent of lactide and glycolide component to statin, about 80:20 weight percent of lactide and glycolide component to statin, about 85:15 weight percent of lactide and glycolide component to statin, about 90:10 weight percent of lactide and glycolide component to statin, and about 99:1 weight percent of lactide and glycolide component to statin.

Generally with respect to the lactide and the glycolide component of the terpolymer, the lactide ranges from about 1 to about 99 weight percent with the amount of glycolide ranging from about 99 to about 1 weight percent. Thus, the lactide and glycolide component ranges from about 1:99 weight percent of lactide to glycolide, about 10:90 weight percent of lactide to glycolide, about 20:80 weight percent of lactide to glycolide, about 30:70 weight percent of lactide to glycolide, about 40:60 weight percent of lactide to glycolide, about 50:50 weight percent of lactide to glycolide, about 60:40 weight percent of lactide to glycolide, about 70:30 weight percent of lactide to glycolide, about 75:25 weight percent of lactide to glycolide, about 80:20 weight percent of lactide to glycolide, about90 weight percent of lactide to glycolide, and about 99:1 weight percent of lactide to glycolide.

A preferred polylactide glycolide statin terpolymer comprises 70 weight percent lactide, 15 weight percent glycolide and 15 weight percent statin in the absence of any epsilon-caprolactone.

Polylactide Lactone Statin Terpolymer. Generally, the lactide and lactone component ranges from about 1 to about 99 weight percent with the amount of the statin ranging from about 99 to about 1 weight percent. The polylactide lactone statin terpolymer does not contain any glycolide. Thus, the polylactide lactone statin terpolymer ranges from about 1:99 weight percent of lactide and lactone component to statin, about 10:90 weight percent of lactide and lactone component to statin, about 20:80 weight percent of lactide and lactone component to statin, about 30:70 weight percent of lactide and lactone component to statin, about 40:60 weight percent of lactide and lactone component to statin, about 50:50 weight percent of lactide and lactone component to statin, about 60:40 weight percent of lactide and lactone component to statin, about 70:30 weight percent of lactide and lactone component to statin, about 80:20 weight percent of lactide and lactone component to statin, about 85:15 weight percent of lactide and lactone component to statin, about 90:10 weight percent of lactide and lactone component to statin, and about 99:1 weight percent of lactide and lactone component to statin.

Generally with respect to the lactide and lactone component, the lactide ranges from about 1 to about 99 weight percent with the lactone ranging from about 99 to about 1 weight percent. Thus, the lactide and lactone component ranges from about 1:99 weight percent of lactide to lactone, about 10:90 weight percent of lactide to lactone, about 20:80 weight percent of lactide to lactone, about 30:70 weight percent of lactide to lactone, about 40:60 weight percent of lactide to lactone, about 50:50 weight percent of lactide to lactone, about 60:40 weight percent of lactide to lactone, about 70:30 weight percent of lactide to lactone, about 75:25 weight percent of lactide to lactone, about 80:20 weight percent of lactide to lactone, about 90:10 weight percent of lactide to lactone, and about 99:1 weight percent of lactide to lactone.

A preferred lactide lactone-statin terpolymer comprises 70 weight percent lactide, 15 weight percent lactone and 15 weight percent statin in the absence of any glycolide.

Polylactide Epsilon-Caprolactone Statin Terpolymer. Generally, the lactide and epsilon-caprolactone component ranges from about 1 to about 99 weight percent with the amount of the statin ranging from about 99 to about 1 weight percent. The polylactide epsilon-caprolactone statin terpolymer does not contain any glycolide. Thus, the polylactide epsilon-caprolactone statin terpolymer ranges from about 1:99 weight percent of lactide and epsilon-caprolactone component to statin, about 10:90 weight percent of lactide and epsilon-caprolactone component to statin, about 20:80 weight percent of lactide and epsilon-caprolactone component to statin, about 30:70 weight percent of lactide and epsilon-caprolactone component to statin, about 40:60 weight percent of lactide and epsilon-caprolactone component to statin, about 50:50 weight percent of lactide and epsilon-caprolactone component to statin, about 60:40 weight percent of lactide and epsilon-caprolactone component to statin, about 70:30 weight percent of lactide and epsilon-caprolactone component to statin, about 80:20 weight percent of lactide and epsilon-caprolactone component to statin, about 85:15 weight percent of lactide and epsilon-caprolactone component to statin, about 90:10 weight percent of lactide and epsilon-caprolactone component to statin, and about 99:1 weight percent of lactide and epsilon-caprolactone component to statin.

Generally with respect to the lactide and epsilon-caprolactone component, the lactide ranges from about 1 to about 99 weight percent with the epsilon-caprolactone ranging from about 99 to about 1 weight percent. Thus, the lactide and epsilon-caprolactone component ranges from about 1:99 weight percent of lactide to epsilon-caprolactone, about 10:90 weight percent of lactide to epsilon-caprolactone, about 20:80 weight percent of lactide to epsilon-caprolactone, about 30:70 weight percent of lactide to epsilon-caprolactone, about 40:60 weight percent of lactide to epsilon-caprolactone, about 50:50 weight percent of lactide to epsilon-caprolactone, about 60:40 weight percent of lactide to epsilon-caprolactone, about 70:30 weight percent of lactide to epsilon-caprolactone, about 75:25 weight percent of lactide to epsilon-caprolactone, about 80:20 weight percent of lactide to epsilon-caprolactone, about 90:10 weight percent of lactide to epsilon-caprolactone, and about 99:1 weight percent of lactide to epsilon-caprolactone.

A preferred lactide epsilon-caprolactone-statin terpolymer comprises 70 weight percent lactide, 15 weight percent epsilon-caprolactone and 15 weight percent statin in the absence of any glycolide.

Polyglycolide Lactone Statin Terpolymer. Generally, the glycolide and lactone component ranges from about 1 to about 99 weight percent with the statin component of the terpolymer ranging from about 99 to about 1 weight percent. The polyglycolide lactone statin terpolymer does not contain any lactide. Thus, the glycolide lactone statin terpolymer ranges from about 1:99 weight percent of glycolide and lactone component to statin, about 10:90 weight percent of glycolide and lactone component to statin, about 20:80 weight percent of glycolide and lactone component to statin, about 30:70 weight percent of glycolide and lactone component to statin, about 40:60 weight percent of glycolide and caprolactone component to statin, about 50:50 weight percent of glycolide and caprolactone component to statin, about 60:40 weight percent of glycolide and lactone component to statin, about 70:30 weight percent of glycolide and lactone component to statin, about 80:20 weight percent of glycolide and lactone component to statin, about 85:15 weight percent of glycolide and lactone component to statin, about 90:10 weight percent of glycolide and lactone component to statin, and about 99:1 weight percent of glycolide and lactone component to statin.

Generally with respect to the glycolide and lactone component of the glycolide lactone statin terpolymer, the glycolide may range from about 1 to about 99 weight percent with the lactone ranging from about 99 to about 1 weight percent. Thus, the glycolide and lactone component ranges from about 1 weight percent of glycolide to 99 weight percent of lactone, about 10:90 weight percent of glycolide to lactone, about 20:80 weight percent of glycolide to lactone, about 30:70 weight percent of glycolide to lactone, about 40:60 weight percent of glycolide to lactone, about 50:50 weight percent of glycolide to lactone, about 60:40 weight percent of glycolide to lactone, about 70:30 weight percent of glycolide to lactone, about 75:25 weight percent of glycolide to lactone, about 80:20 weight percent of glycolide to lactone, about 90:10 weight percent of glycolide to lactone, and about 99:1 glycolide to lactone.

A preferred polyglycolide lactone statin terpolymer comprises 70 weight percent glycolide, 15 weight percent lactone and 15 weight percent statin.

Polyglycolide Epsilon-Caprolactone Statin Terpolymer. Generally, the glycolide and epsilon-caprolactone component ranges from about 1 to about 99 weight percent with the statin component of the terpolymer ranging from about 99 to about 1 weight percent. The polyglycolide epsilon-caprolactone statin terpolymer does not contain any lactide. Thus, the glycolide epsilon-caprolactone statin terpolymer ranges from about 1:99 weight percent of glycolide and epsilon-caprolactone component to statin, about 10:90 weight percent of glycolide and epsilon-caprolactone component to statin, about 20:80 weight percent of glycolide and epsilon-caprolactone component to statin, about 30:70 weight percent of glycolide and epsilon-caprolactone component to statin, about 40:60 weight percent of glycolide and caprolactone component to statin, about 50:50 weight percent of glycolide and caprolactone component to statin, about 60:40 weight percent of glycolide and epsilon-caprolactone component to statin, about 70:30 weight percent of glycolide and epsilon-caprolactone component to statin, about 80:20 weight percent of glycolide and epsilon-caprolactone component to statin, about 85:15 weight percent of glycolide and epsilon-caprolactone component to statin, about 90:10 weight percent of glycolide and epsilon-caprolactone component to statin, and about 99:1 weight percent of glycolide and epsilon-caprolactone component to statin.

Generally with respect to the glycolide and epsilon-caprolactone component of the glycolide epsilon-caprolactone statin terpolymer, the glycolide may range from about 1 to about 99 weight percent with the epsilon-caprolactone ranging from about 99 to about 1 weight percent. Thus, the glycolide and epsilon-caprolactone component ranges from about 1 weight percent of glycolide to 99 weight percent of epsilon-caprolactone, about 10:90 weight percent of glycolide to epsilon-caprolactone, about 20:80 weight percent of glycolide to epsilon-caprolactone, about 30:70 weight percent of glycolide to epsilon-caprolactone, about 40:60 weight percent of glycolide to epsilon-caprolactone, about 50:50 weight percent of glycolide to epsilon-caprolactone, about 60:40 weight percent of glycolide to epsilon-caprolactone, about 70:30 weight percent of glycolide to epsilon-caprolactone, about 75:25 weight percent of glycolide to epsilon-caprolactone, about 80:20 weight percent of glycolide to epsilon-caprolactone, about 90:10 weight percent of glycolide to epsilon-caprolactone, and about 99:1 glycolide to epsilon-caprolactone.

A preferred polyglycolide epsilon-caprolactone statin terpolymer comprises 70 weight percent glycolide, 15 weight percent epsilon-caprolactone and 15 weight percent statin.

Finally, it should be noted that oral statin dosing as described above will not be changed by this invention. Statins delivered locally by this scaffold will be resorbed by the vessel wall and will not have systemic effects. Vulnerable plaques are distributed throughout the coronary artery tree and the ones that are most prone to rupture and cause initial thrombus formation are the ones in arteries that are minimally narrowed. These lesions are generally not candidates for stenting. For these lesions oral systemic statin therapy is crucial. But for those lesions that are stentable, statins delivered locally at the site of the injury produced by the procedure are needed to promote endothelial regrowth and to offset the effect of drugs eluted from polymers coated onto the surface of scaffolds that prevent restenosis but cause endothelial dysfunction, inhibit re-endothelialization, increase the risk of stent thrombosis and prolong the requirement for dual antiplatelet therapy and risk of bleeding following stent placement.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

What is claimed is:

1. A polymer comprising NOS agonist and one or more monomers selected from the group consisting of lactide, glycolide, and epsilon caprolactone, wherein the NOS agonist is incorporated as repeating monomer units into the body of the polymer.

2. The polymer of claim 1, wherein the NOS agonist comprises one or more carboxylic acid groups and one or more alkylhydroxy groups.

3. The polymer of claim 1, wherein the NOS agonist comprises a carboxylic acid group and an alkylhydroxy group that can be joined to form a lactone containing cyclic ring.

4. The polymer of claim 1, wherein the NOS agonist comprises HMG CoA reductase inhibitor or statin.

5. The polymer of claim 4, wherein the statin is selected from the group consisting of velostatin, dihydrocompactin, carvastatin, bevastatin, cefvastatin, glenvastatin, simvastatin, lovastatin, atorvastatin, pravastatin, cerivastatin, rosuvastatin, pitavastatin, fluvastatin, mevastatin, dalvastatin, compactin and mixtures thereof.

6. The polymer of claim 5, wherein the statin is selected from the group consisting of simvastatin, lovastatin, atorvastatin, pravastatin, cerivastatin, rosuvastatin, pitavastatin, fluvastatin, mevastatin, dalvastatin, compactin, and mixtures thereof.

7. The polymer of claim 1, wherein the polymer is selected from the group consisting of polylactide NOS agonist, polyglycolide NOS agonist, polyepsilon caprolactone NOS agonist, polylactideglycolide NOS agonist, polylactideepsilon caprolactone NOS agonist, polyglycolideepsilon caprolactone NOS agonist, polylactideglycolide epsiloncaprolactone NOS agonist and mixtures thereof.

8. The polymer of claim 7, wherein the NOS agonist comprises one or more carboxylic acid groups and one or more alkylhydroxy groups.

9. The polymer of claim 7, wherein the NOS agonist comprises a carboxylic acid group and an alkylhydroxy group that can be joined to form a lactone containing cyclic ring.

10. The polymer of claim 7, wherein the NOS agonist comprises HMG CoA reductase inhibitor or statin.

11. The polymer of claim 10, wherein the statin is selected from the group consisting of velostatin, dihydrocompactin, carvastatin, bevastatin, cefvastatin, glenvastatin, simvastatin, lovastatin, atorvastatin, pravastatin, cerivastatin, rosuvastatin, pitavastatin, fluvastatin, mevastatin, dalvastatin, compactin and mixtures thereof.

12. The polymer of claim 11, wherein the statin is selected from the group consisting of simvastatin, lovastatin, atorvastatin, pravastatin, cerivastatin, rosuvastatin, pitavastatin, fluvastatin, mevastatin, dalvastatin, compactin, and mixtures thereof.

13. A polymer comprising NOS agonist, lactide, glycolide, and lactone, wherein the NOS agonist is incorporated as repeating monomer units into the body of the polymer.

14. The polymer of claim 13, wherein the NOS agonist comprises one or more carboxylic acid groups and one or more alkylhydroxy groups.

15. The polymer of claim 13, wherein the NOS agonist comprises a carboxylic acid group and an alkylhydroxy group that can be joined to form a lactone containing cyclic ring.

16. The polymer of claim 13, wherein the NOS agonist comprises HMG CoA reductase inhibitor or statin.

17. The polymer of claim 16, wherein the statin is selected from the group consisting of velostatin, dihydrocompactin, carvastatin, bevastatin, cefvastatin, glenvastatin, simvastatin, lovastatin, atorvastatin, pravastatin, cerivastatin, rosuvastatin, pitavastatin, fluvastatin, mevastatin, dalvastatin, compactin and mixtures thereof.

18. The polymer of claim 17, wherein the statin is selected from the group consisting of simvastatin, lovastatin, atorvastatin, pravastatin, cerivastatin, rosuvastatin, pitavastatin, fluvastatin, mevastatin, dalvastatin, compactin, and mixtures thereof.
